(19) United States
(12) Patent Application Publication
(10) Pub. No.: US 2007/0118092 A1
(43) Pub. Date: May 24, 2007

(54) Disposable, absorbent panties

(76) Inventor: Angelica Blanco, Hialeah, FL (US)

Correspondence Address: LITMAN LAW OFFICES, LTD., P.O. BOX 15035, CRYSTAL CITY STATION, ARLINGTON, VA, 22215, US

(21) Appl. No.: 11/283,886

(22) Filed: Nov. 22, 2005

(51) Int. Cl. A61F 13/15

(52) U.S. Cl. 604/396

(57) ABSTRACT

The disposable absorbent panties is a thong panty having a liner in the crotch area that incorporates an absorbent insert. The insert is a tampon adapted to penetrate slightly into the vagina to absorb vaginal discharges. The edges of the panty are elasticized so that the panty will fit tightly around the user's legs, thereby preventing any possible leakage. A thong-type panty assures a comfortable fit and decreases the amount of material and cost needed to manufacture the panty. The materials used are biodegradable for proper ecological disposal.

### BACKGROUND OF THE INVENTION

1. Field of the Invention

The present invention generally relates to intimate wearing apparel. More specifically, the present invention is drawn to a disposable panty that incorporates a tampon and a moisture absorbing liner.

2. Description of the Related Art

From around thirteen years of age until menopause, the monthly menstrual cycle presents at least a mild inconvenience in a woman's daily routine in that extra care must be taken to assure proper hygienic protection. To this end, an array of feminine care products is available for use during the menstrual period. These products include sanitary napkins, lined panties, vaginal inserts and the like. These products, while somewhat satisfactory, still leave something to be desired for the “today's woman”. The art would certainly welcome a hygienic device that incorporates ease of use, comfort, superior protection and convenient disposal.

Thus, disposable absorbent panties solving the aforementioned problems is desired.

### SUMMARY OF THE INVENTION

The present invention is drawn to a disposable, absorbent, thong-type panty. The thong panty includes a liner in the crotch area that incorporates an absorbent vaginal insert and pad. The insert is a tampon adapted to penetrate slightly into the vagina to absorb vaginal discharges. The edges of the panty are elasticized so that the panty will fit tightly around the user's legs, thereby preventing any possible leakage. A thong-type panty assures a comfortable fit and decreases the amount of material and cost needed to manufacture the panty. The materials used will be biodegradable for proper ecological disposal. As contemplated, the panty will be manufactured in a variety of colors.

Accordingly, the disposable absorbent panties provide a disposable undergarment that is efficient and can be comfortably worn by females during the menstrual period.

These and other features of the present invention will become readily apparent upon further review of the following specification and drawings.

### BRIEF DESCRIPTION OF THE DRAWINGS

FIG. 1 is an environmental, perspective view of a disposable, absorbent, thong-type panty according to the present invention.

FIG. 2 is a perspective view of a disposable, absorbent, thong-type panty according to the present invention.

FIG. 3 is a partial sectional view of the crotch area of a disposable, thong-type panty according to the present invention.

Similar reference characters denote corresponding features consistently throughout the attached drawings.

### DETAILED DESCRIPTION OF THE PREFERRED EMBODIMENT

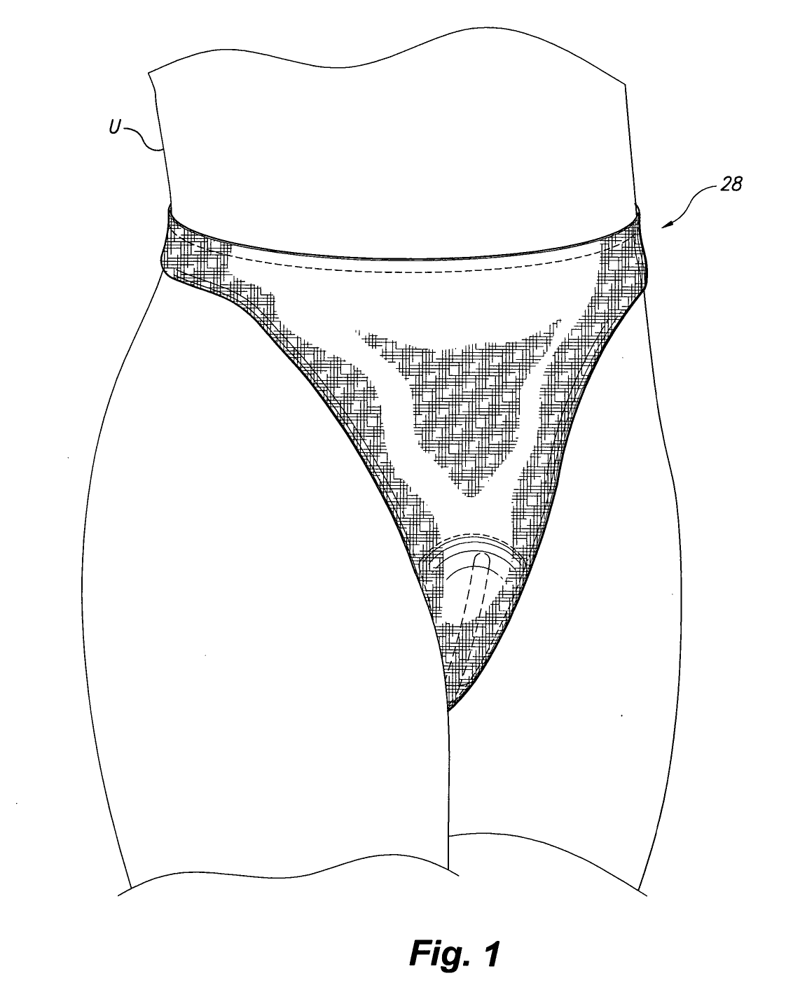

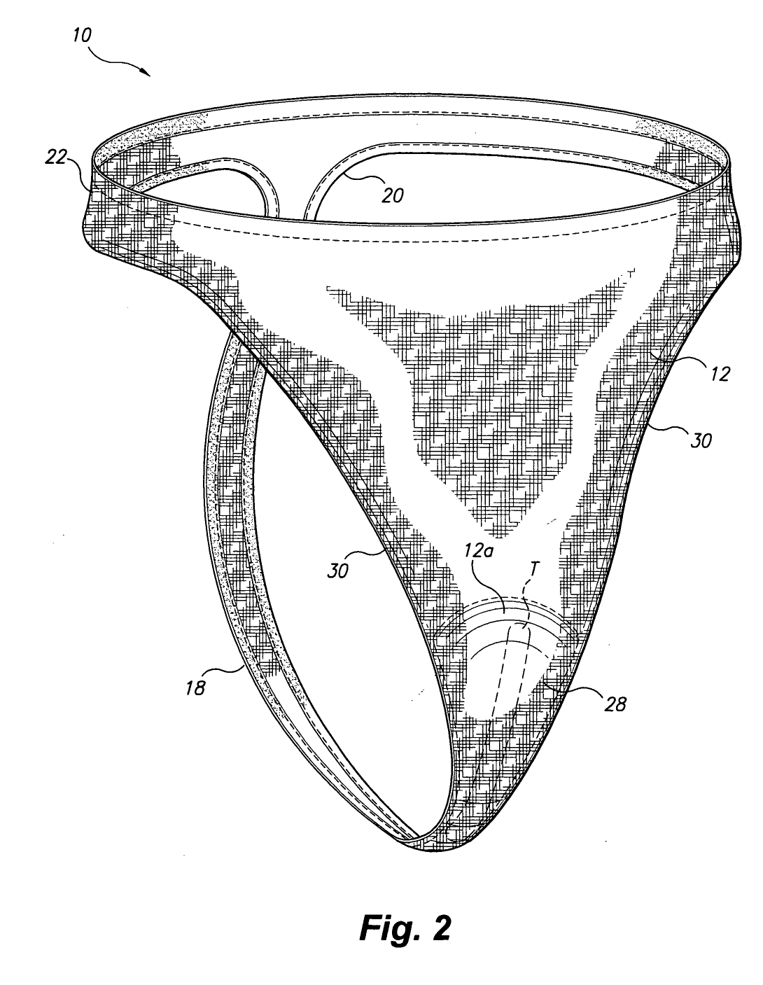

Attention is directed to FIGS. 1-3 wherein the disposable absorbent panties of the present invention is generally indicated at 10. Panty 10 is of a one-piece design and comprises a front triangular-shaped portion 12 that includes a crotch portion 12a. Front portion 12 is provided with an inner surface 14 and an outer surface 16. Front portion 12 tapers from a relative wide upper end to a narrower lower end. A triangular shaped rear portion 20 is fashioned with a relatively wide upper end and tapers to a narrower lower end. A narrow thong 18 extends from the lower end of front portion 12 and merges into the lower end of triangular-shaped rear portion 20. A continuous narrow waistband 22 connects the upper end of front portion 12 with the upper end of rear portion 20.

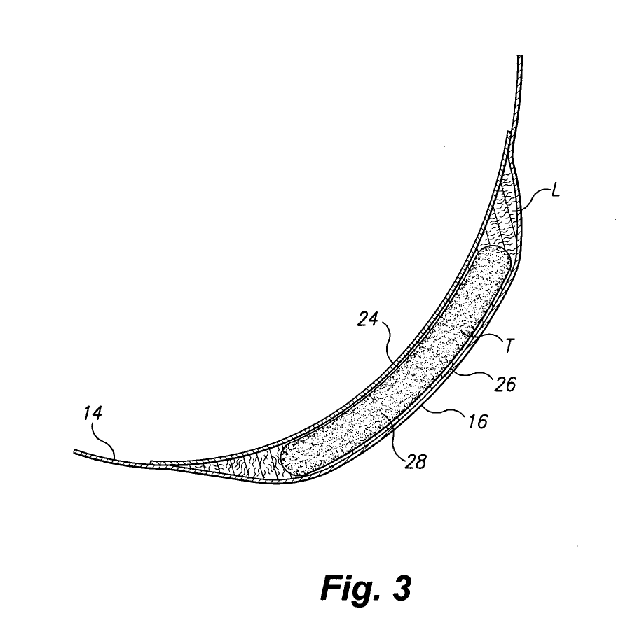

As best seen in FIG. 3, inner surface 14 of crotch portion 12a incorporates a liner having a layered arrangement that includes a first layer 24 and a second layer 26. A vaginal insert member (tampon) 28 is disposed between layers 24 and 26. Vaginal insert member 28 extends upwardly from inner surface 14 and is of a thickness to protrude into the vagina when the panty is donned by a user U. Layer 24 is fabricated from a liquid-permeable material, while layer 26 and vaginal insert member 28 are fabricated from liquid-absorbent materials. The material that forms the outer surface 16 is fabricated from a liquid-impermeable material. An elastic material 30 forms the edges of the one-piece panty to assure a secure fit at the legs in the crotch area. As indicated above, the secure fit at the legs will insure that no leakage will occur.

It is to be understood that the present invention is not limited to the embodiment described above, but encompasses any and all embodiments within the scope of the following claims.

I claim:

1. A disposable absorbent panty, comprising:
a front triangular-shaped member having a relatively wide upper end tapering to a narrower lower end, the front triangular-shaped member having an inner surface, an outer surface and a crotch area;
a rear triangular-shaped member having a relatively wide upper end tapering to a relatively narrow lower end;
a narrow thong connecting a first portion of the front triangular-shaped member to a first portion of the rear triangular-shaped member;
a continuous waistband connecting a second portion of the front triangular-shaped member to a second portion of the rear triangular-shaped member; and
a liner disposed on the inner surface of said front triangular-shaped portion, the liner having a vaginal insert member disposed therein.

2. The disposable absorbent panty according to claim 1, wherein said liner further includes a first layer and a second layer, and wherein said vaginal insert member comprises a tampon disposed between said first layer and said second layer.

3. The disposable absorbent panty according to claim 1, wherein said panty is completely fabricated from biodegradable material.

4. The disposable absorbent panty according to claim 1, wherein the outer surface of said front triangular-shaped member is fabricated from liquid impermeable material.

5. The disposable absorbent panty according to claim 1, wherein said vaginal insert member is fabricated from a liquid absorbent material.

6. The disposable absorbent panty according to claim 1, wherein said front triangular-shaped member has edges formed with elasticized material.

7. A disposable absorbent panty, comprising:
a front triangular-shaped member having a relatively wide upper end tapering to a narrower lower end, the front triangular-shaped member having an inner surface, an outer surface and a crotch area;
a rear triangular-shaped member having a relatively wide upper end tapering to a relatively narrow lower end;
a narrow thong connecting the lower end of the front triangular-shaped member to the lower end of the rear triangular-shaped member;
a continuous waistband connecting the upper end of the front triangular-shaped member to the upper end of the rear triangular-shaped member; and
a liner disposed on the inner surface of the front triangular-shaped portion, the liner having a vaginal insert member disposed therein.

8. The disposable absorbent panty according to claim 7, wherein said liner further includes a first layer and a second layer, and wherein said vaginal insert member comprises a tampon disposed between said first layer and said second layer.

9. The disposable absorbent panty according to claim 7, wherein said panty is completely fabricated from biodegradable material.

10. The disposable absorbent panty according to claim 7, wherein the outer surface of said front triangular-shaped member is fabricated from liquid impermeable material.

11. The disposable absorbent panty according to claim 7, wherein said vaginal insert member is fabricated from a liquid absorbent material.

12. The disposable absorbent panty according to claim 7, wherein said front triangular-shaped member has edges formed with elasticized material.

13. A disposable absorbent panty, comprising:
a front triangular-shaped member having a relatively wide upper end tapering to a narrower lower end, the front triangular-shaped member having an inner surface, an outer surface and a crotch area;
a rear triangular-shaped member having a relatively wide upper end tapering to a relatively narrow lower end;
a narrow thong connecting the lower end of the front triangular-shaped member to the lower end of the rear triangular-shaped member;
a continuous waistband connecting the upper end of the front triangular-shaped member to the upper end of the rear triangular-shaped member; and
a liner disposed on the inner surface of the front triangular-shaped portion at the crotch area, the liner having a vaginal insert member disposed therein, the vaginal insert member being a tampon fabricated from a liquid absorbent material.

14. The disposable absorbent panty according to claim 13, wherein said liner further includes a first layer fabricated from a liquid permeable material and a second layer fabricated from a liquid absorbent material, said vaginal insert member being disposed between said first layer and said second layer.

15. The disposable absorbent panty according to claim 14, wherein said front triangular-shaped member has edges formed with elasticized material.

16. The disposable absorbent panty according to claim 15, wherein said panty is completely fabricated from biodegradable material.
